(19) United States
(12) Patent Application Publication
(10) Pub. No.: US 2021/0186862 A1
(43) Pub. Date: Jun. 24, 2021

(54) METHOTREXATE TOPICAL SOLUTION FOR TREATMENT OF PSORIASIS

(71) Applicant: SHILPA MEDICARE LIMITED, RAICHUR (IN)

(72) Inventors: MOHAN KUMAR BHANDARI, Bangalore (IN); SREENIVASA REDDY, Bangalore (IN); SHIVAKUMAR PRADEEP, Vizianagaram (IN); THIPPESWAMY AGADIHIREMATH, Bangalore (IN)

(73) Assignee: SHILPA MEDICARE LTD, RAICHUR (IN)

(21) Appl. No.: 16/762,499

(22) PCT Filed: Dec. 4, 2018
(86) PCT No.: PCT/IB2018/059592

§ 371 Date: May 8, 2020

(30) Foreign Application Priority Data
Dec. 5, 2017 (IN) 201741043541

(51) Int. Cl. A61K 9/00; A61K 31/519; A61P 17/06; A61K 47/36; A61K 47/06; A61K 9/06; A61K 9/08

(52) U.S. Cl. CPC A61K 9/0014; A61K 31/519; A61P 17/06; A61K 9/08; A61K 47/06; A61K 9/06; A61K 47/36

(57) ABSTRACT

The present invention relates to a topical pharmaceutical composition containing of about 0.01% w/w to about 0.1% w/w of methotrexate in topical solution form for external use for the treatment of psoriasis. The present invention also further encompasses the process for the preparation of the methotrexate in topical solution form.

### FIELD OF THE INVENTION

The present invention relates to topical pharmaceutical composition comprising of about 0.01% w/w to about 0.5% w/w of methotrexate for the treatment of psoriasis. It relates more specifically topical pharmaceutical solution containing methotrexate.

### BACKGROUND OF THE INVENTION

Methotrexate is an antimetabolite of the antifolate type, is approved by the FDA as chemotherapy agent and immune system suppressant.

Methotrexate (MTX) is an effective agent in the treatment of psoriasis, including pustular psoriasis, psoriatic erythroderma, psoriatic arthritis and for extensive chronic plaque psoriasis not controlled by conventional therapy.

Methotrexate is usually reserved for patients with moderate to severe disease who have at least 5% of their skin covered with psoriasis who are not responsive to, or eligible for, topical or ultraviolet light treatments (including UVB and PUVA).

Chemically methotrexate is N-[4-[[(2,4-diamino-6-pteridinyl) methyl] methylamino] benzoyl]-L-glutamic acid, with molecular weight: 454.45 and is structurally represented as

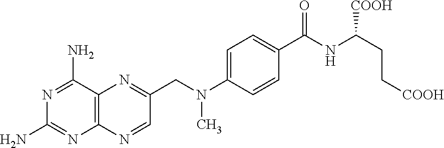 [SMILES: CN(Cc1cnc2nc(N)nc(N)c2n1)c1ccc(C(=O)N[C@@H](CCC(=O)O)C(=O)O)cc1]

Methotrexate inhibits dihydrofolate reductase (DHFR), which is required to produce tetrahydrofolic acid, the active from of folate in humans. Folate is essential for purine and pyrimidine synthesis and thus for the replication of DNA. Methotrexate acts on this enzyme binding to it some 1000 times more tightly than folate itself resulting in a substantial negative effect on rapidly dividing cells, including cancer cells.

Inhibition of DHFR is more relevant to high dose MTX regimens used in cancer therapy, however low dose MTX therapy appears to inhibit enzymes involved in purine metabolism, leading to an accumulation of adenosine, which has anti-inflammatory properties and subsequent immunosuppression through adenosine receptors.

Binding of adenosine to A2 and A3 receptors results in a favourable situation which is probably one of the important anti-inflammatory mechanisms of MTX action. MTX inhibits T cell activation and suppresses the intercellular adhesion molecule expression by T cells. Interleukin-1 (IL-1), a critical inflammatory cytokine, has some structural similarity to DHFR and it appears to inhibit IL-1 binding to T cell receptors.

Methotrexate acts specifically on the S-phase of the cell cycle. Tissues with a high cellular proliferation rate such as neoplastic tissue, bone marrow, epithelial cells or foetal cells seem to be the most susceptible. Methotrexate is used for this reason in the treatment of psoriasis, where the rate of production of epithelial cells of the skin is much higher than that of normal cells.

Methotrexate may be of value in the symptomatic control of severe, recalcitrant, disabling psoriasis, which is not adequately responsive to other forms of treatment. However, due to the high risk associated with its use, methotrexate should be used after the diagnosis has been definitely established, as by biopsy and/or after dermatologic consultation.

The patient should be fully informed of the risks involved and should be under constant supervision of the physician. Assessment of renal function, liver function and blood elements should be made by history, physical examination and laboratory tests (such as haemogram, urinalysis, serum creatinine, liver function studies and liver biopsy if indicated) before beginning methotrexate, periodically during methotrexate therapy and before reinstituting methotrexate therapy after a rest period. Appropriate steps should be taken to avoid conception for at least 12 weeks following methotrexate therapy.

Delivering methotrexate based medicaments to an affected, local area of a living subject using a systemic delivery method is problematic because of the many severe, sometimes life threatening, side effects associated with systemic delivery of methotrexate based medicaments, such as, for examples, hepatitis, liver fibrosis, cirrhosis, leukopenia (bone marrow suppression), mucositis, ulcerative stomatitis, skin rash, nausea, abdominal distress, malaise, fatigue, chills and fever, diarrhea, gastrointestinal ulceration or perforation, pancreatitis, pericarditis, hypotension, deep venous thrombosis, thrombophlebitis, interstitial pneumonitis, headaches, drowsiness, cognitive dysfunction, reduced immunity, rash, photosensitivity, nephropathy, hematuria, alopecia, defective oogenesis, oligospermia, infertility, miscarriage, and birth defects.

Psoriasis is a long-lasting auto immune disease characterized by patches of abnormal skin. Skin patches are, itchy, and scaly. They may vary in severity from small and localized to complete body coverage. There are five main types of psoriasis: Plaque psoriasis, also known as psoriasis vulgaris, makes up about 90% of cases.

Epidemiology of Psoriasis:
Psoriasis is an ancient chronic skin disease.
Uncommon under the age of 10 years, it appears between the ages of 15 and 30 years affecting men and women.
Psoriasis is universal in occurrence, but its prevalence in different populations varies from 0.1% to 11.8%.
In fact, this pathology shows a significant geographical variability with the lowest incidence seen at the equator and increasing frequency towards the poles.

Many medications have been discovered for the treatment of psoriasis, with methotrexate and cyclosporine being the earliest treatment options. They are complex structures that have shown efficacy, but both carry possible side effects and complications.

Development of Psoriasis: Psoriasis is characterized by an abnormally excessive and rapid growth of the epidermal layer of the skin. Abnormal production of skin cells (especially during wound repair) and an overabundance of skin cells result from the sequence of pathological events in psoriasis. Skin cells are replaced every 3-5 days in psoriasis rather than the usual 28-30 days. These changes are believed to stem from the premature maturation of keratinocytes induced by an inflammatory cascade in the dermis involving dendritic cells, macrophages, and T cells. These immune cells move from the dermis to the epidermis and secrete inflammatory chemical signals (cytokines) such as tumor necrosis factor-α, interleukin-1β, interleukin-6, interleukin-36 and interleukin-22. These secreted inflammatory signals are believed to stimulate keratinocytes to proliferate.

Gene mutations of proteins involved in the skin's ability to function as a barrier have been identified as markers of susceptibility for the development of psoriasis.

DNA released from dying cells acts as an inflammatory stimulus in psoriasis and stimulates the receptors on certain dendritic cells, which in turn produce the cytokine interferon-α. In response to these chemical messages from dendritic cells and T cells, keratinocytes also secrete cytokines such as interleukin-1, interleukin-6, and tumor necrosis factor-α, which signal downstream inflammatory cells to arrive and stimulate additional inflammation.

Treatments for Psoriasis
A broad spectrum of anti-psoriatic treatments, both topical and systemic, is available for the management of psoriasis.
However, it is often resistant to treatment or, else, frequently relapses upon cessation of medication after partial or acceptable clearance is obtained.
The severity of the disease usually determines the therapeutic approach.
Among the treatments, there are topical, phototherapy, biologic and systemic treatments. Approximately, 70 to 80% of all patients with psoriasis can be treated adequately with topical therapy.
For others, phototherapy and systemic treatments are effective; however, the duration
of a treatment is restricted because of the cumulative toxicity potential of an individual therapy.

U.S. Pat. No. 8,067,376 discloses the method for transdermally delivering methotrexate and protein transduction domain (PTD) for the treatment of autoimmune disease like psoriasis or rheumatoid arthritis.

U.S. Pat. No. 6,485,740 discloses a methotrexate containing transdermal preparation effective for rheumatoid arthritis comprises an organic amine. The organic amine may preferably be an alkonolamine such as monoethanolamine, diethanolamine, triethanolamine or diisopropanolamine, or an alkylamine such as ethylamine, diethylamine or triethylamine. The transdermal preparation is, for example, a plaster, a cataplasm, an ointment, a cream or a lotion. The base of this product is non aqueous but an oil based formulation containing an organic amine.

US application No. 20150079175 discloses the topical pharmaceutical composition in the form of a cream, cream-gel or lotion, in an oil/water emulsion comprising a combination of methotrexate monohydrate, bisabolol and allantoin.

US Application 20050153969 discloses the method for treating neoplastic, angiogenic, fibroblastic, and/or immunosuppressive ocular irregularities of a living subject, comprising the steps of: providing a living subject, wherein the living subject includes an affected ocular area having a neoplastic, angiogenic, fibroblastic, and/or immunosuppressive irregularity; providing a methotrexate based medicament, wherein the methotrexate based medicament is capable of inhibiting DNA synthesis; associating a therapeutically effective concentration of the methotrexate based medicament with the affected ocular area of the living subject; and decreasing the neoplastic, angiogenic, fibroblastic, and/or immunosuppressive ocular irregularity of the living subject. This formulation does not suggest methotrexate topical application.

Indian Patent No. 207193 discloses the Methotrexate topical semisolid dosage form (gel) comprising, therapeutically effective amounts of Methotrexate, and a gel forming agent, penetration enhancing agent, preservative, fragrance, surfactant and pharmaceutically acceptable excipients such as solvents and pH adjusting agents to adjust the pH between 6.8 and 7.4. The quantity of methotrexate used in the inventive composition of IN '193 patent contains methotrexate in range from about 0.1% w/w to about 1.5% w/w.

However, there exists a need to develop a topical pharmaceutical composition comprising of about 0.01% w/w to about 0.1% w/w of methotrexate for treatment of psoriasis that further reduces side effects, premedication requirements, and patient noncompliance issues associated with the currently marketed formulations.

### OBJECT OF THE INVENTION

The object of the invention is to provide a topical pharmaceutical composition comprising of about 0.01% w/w to about 0.1% w/w of methotrexate.

Another object of the invention is to provide a topical pharmaceutical composition containing of about 0.01% w/w to about 0.1% w/w of methotrexate in topical solution form for external use for the treatment of psoriasis.

### SUMMARY OF THE INVENTION

According to the invention, the present invention provides a topical pharmaceutical composition comprising of about 0.01% w/w to about 0.1% w/w of methotrexate.

The topical pharmaceutical composition of present invention is present in the dosage forms of solution, ointment, cream, gel and paste.

In one embodiment of the invention the present invention provides a topical pharmaceutical composition comprising of about 0.01% w/w to about 0.1% w/w of methotrexate, wherein the composition is in solution form.

In another embodiment, the present invention provides a topical pharmaceutical composition comprising of about 0.01% w/w to about 0.1% w/w of methotrexate, about 0.1% w/w to about 1.0% w/w of polymer and pharmaceutically acceptable excipients.

In a further embodiment, the present invention provides a topical pharmaceutical composition comprising of about 0.01% w/w to about 0.1% w/w of methotrexate, about 0.1% w/w to about 1.0% w/w of xanthan gum and pharmaceutically acceptable excipients.

In another embodiment, the present invention provides a topical pharmaceutical solution comprising of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents.

In still further embodiment, the present invention provides a topical pharmaceutical solution comprising of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents to adjust the pH of about 7.0.

### BRIEF DESCRIPTION OF DRAWINGS

FIG. 1 depicts the psoriasis score of seven days of example 3, 4, 5 and 6.

FIG. 2 depicts the psoriasis score of fourteen days of example 3, 4, 5 and 6.

FIG. 3 depicts the psoriasis score of seven days of example 13, 14, 15 and 16.

FIG. 4 depicts the psoriasis score of fourteen days of example 13, 14, 15 and 16.

### DETAILED DESCRIPTION OF THE INVENTION

As used herein in connection with numerical values, the terms “about” mean+/−10% of the indicated value, including the indicated value.

The present invention provides herein, a topical pharmaceutical composition comprising of about 0.01% w/w to about 0.1% w/w of methotrexate which will be used to treatment of psoriasis.

In embodiments of the invention, the topical pharmaceutical composition of the present invention comprises methotrexate as the active ingredient from about 0.01% w/w to about 0.1% w/w, preferably from 0.01% w/w to about 0.099% w/w, and more preferably 0.01% w/w, 0.015% w/w, 0.02% w/w, 0.025% w/w, 0.03% w/w, 0.035% w/w, 0.04% w/w, 0.045% w/w, 0.05% w/w, 0.055% w/w, 0.06% w/w, 0.065% w/w, 0.07% w/w, 0.075% w/w, 0.08% w/w, 0.085% w/w, 0.09% w/w, 0.095% w/w and 0.99% w/w based on the total weight of the composition.

In embodiments of the invention, the topical pharmaceutical composition of present invention is present in the dosage forms of solution, ointment, cream, gel and paste and in a preferred embodiment, the topical pharmaceutical composition of the invention is solution dosage form.

In another embodiment, the present invention provides a topical pharmaceutical composition comprising about 0.01% w/w to about 0.1% w/w of methotrexate, polymer (viscosity modifiers) and pharmaceutically acceptable excipients.

Examples of the viscosity modifier (polymers) are selected from the group consisting of carboxylated vinyl polymers such as polyacrylic acids and sodium salts thereof, boric acid, diethanolamide, coco-diethanolamide, coco-monoethanolamide, stearic-diethanolamide, ethoxylated cellulose, hydroxyethyl styrylamide, oleic-diethanolamide, stearic-monoethanolamide, cetyl alcohol, steroyl alcohol, polyacrylamide thickeners, ethanol glycol disterate, xanthan compositions (xanthan gum), sodium alginate andalgin products, hydroxypropyl cellulose and hydroxyethyl cellulose. Preferably, the viscosity modifier selected is xanthan gum. Viscosity modifier preferably used in the pharmaceutical topical solution composition is of about 0.1% w/w to about 5% w/w based on the total weight of the composition. More preferably, the viscosity modifier used in the composition is of about 0.1% w/w to about 1.0% based on total weight of the composition and most preferably of about 0.3% w/w of total weight of composition.

In another embodiment, the present invention provides a topical pharmaceutical composition comprising about 0.01% w/w to about 0.1% w/w of methotrexate, 0.1% w/w to about 1% w/w of polymer (viscosity modifiers) and pharmaceutically acceptable excipients.

In another embodiment, the present invention provides a topical pharmaceutical composition consisting essentially of about 0.01% w/w to about 0.1% w/w of methotrexate, 0.1% w/w to about 1% w/w of polymer (viscosity modifiers) and pharmaceutically acceptable excipients.

In another embodiment, the present invention provides a topical pharmaceutical composition comprising of about 0.01% w/w to about 0.1% w/w of methotrexate, 0.1% w/w to about 1% w/w of xanthan gum and pharmaceutically acceptable excipients.

In still another embodiment, the present invention provides a topical pharmaceutical composition consisting essentially of about 0.01% w/w to about 0.1% w/w of methotrexate, 0.1% w/w to about 1% w/w of xanthan gum and pharmaceutically acceptable excipients.

In embodiments of the invention the pharmaceutically acceptable excipients used in the invention are selected from the group consisting of solubilizers, buffering agents, antioxidants, preservatives, pH adjusting agents and solvents.

In embodiments of the invention solubilizers are selected from the group consisting of glyceryl oleate, isopropyl myristate, methyl laurate, N-lauroyl sarcosine, oleic acid (octadecenoic acid), sodium lauryl sulfoacetate, sodium octyl sulfate, tromethamine, propylene glycol (PG), propylene glycol dipelargonate (PGDP), transcutol, fatty alcohols, such as oleyl alcohol, caprylic alcohol, decyl alcohol, lauryl alcohol, 2-lauryl alcohol, myristyl alcohol, cetyl alcohol, stearyl alcohol, oleyl alcohol, linoleyl alcohol and linolenyl alcohol. Preferably, the solubilizer used in the preferred composition is selected from tromethamine. Solubilizer preferably used in the pharmaceutical topical solution composition is of about 0.01% w/w to about 5% w/w based on the total weight of the composition. More preferably, the solubilizer used in the composition of about 0.05% w/w to about 3% w/w based on total weight of the composition and most preferably solubilizer used is of about 0.3% w/w of the total weight of topical solution.

In embodiments of the invention, buffering agents are selected from the group consisting of diethanolamine, triethanolamine, sodium hydroxide, hydrochloric acid, sodium citrate dihydrate, citric acid and mono basic sodium phosphate. Buffering agent preferably used in the present invention is sodium citrate dihydrate. Buffering agent preferably used in the present composition is of about 0.01% w/w to about 2% w/w based on the total weight of the composition. More preferably buffering agent used in the present composition is of about 0.1% w/w to about 1% w/w based on total weight of composition and most preferably of about 0.2% w/w based on total weight of total weight of composition.

In embodiments of the invention antioxidants are selected from the group consisting of butylated hydroxyanisole (BHA), butylated hydroxytoluene (BHT), ascorbic acid, beta-carotene, alpha-tocopherol, propyl gallate, gentisic acid sodium ascorbate, sodium bisulfite, sodium metabisulfite, monothioglycerol, cysteine, thioglycolate sodium, acetone sodium bisulfite, ascorbate (sodium/acid), bisulfite sodium, cystein/cysteinate HCl, dithionite sodium (Na hydrosulfite, Na sulfoxylate), gentisic acid, gentisic acid ethanolamine, glutamate monosodium, formaldehyde sulfoxylate sodium, metabisulfite potassium, metabisulfite sodium, monothioglycerol (thioglycerol), propyl gallate, sulfite sodium, tocopherol alpha, thioglycolate sodium, EDTA in calcium and sodium compounds (disodium EDTA) or the mixtures thereof. Preferably, the antioxidant is selected from disodium EDTA. Antioxidant preferably used in the pharmaceutical topical solution composition of about 0.001% w/w to about 0.3% w/w based on the total weight of the composition. Most preferably, the antioxidant is used in the composition of about 0.01% w/w to about 0.3% w/w based on total weight of the composition and most preferably 0.025% w/w based on total weight of the composition.

In embodiments of the invention the pH adjusting agents used in the present invention is sodium hydroxide or hydrochloric acid. pH adjusting agent is used to adjust the pH of the present topical composition to about 7.0.

In embodiments of the invention, the topical composition optionally further comprises preservatives. Preservatives used in the present composition is selected from group consisting of methyl paraben, propyl paraben, chlorocresol and benzoyl alcohol.

In embodiments of the invention, the solvents used in present invention are selected from group consisting of purified water, ethanol, methanol, isopropanol or mixtures thereof. The most preferably used solvent in the present invention is purified water.

In another embodiment, the present invention provides a topical pharmaceutical solution comprising of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents.

In still another embodiment, the present invention provides a topical pharmaceutical solution consisting essentially of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents.

In further embodiment, the present invention provides a topical pharmaceutical solution comprising of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents to adjust the pH of about 7.0.

In another embodiment, the present invention provides a topical pharmaceutical solution consisting essentially of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents to adjust the pH of about 7.0.

In one embodiment, the present invention provides a topical pharmaceutical solution comprising of about 0.01% w/w to about 0.1% w/w of methotrexate, about 0.3% w/w of xanthan gum, about 0.3% w/w of tromethamine, about 0.2% w/w of sodium citrate dihydrate, about 0.025% w/w of disodium Edetate, solvents and pH adjusting agents.

In one embodiment, the present invention provides a topical pharmaceutical solution consisting essentially of about 0.01% w/w to about 0.1% w/w of methotrexate, about 0.3% w/w of xanthan gum, about 0.3% w/w of tromethamine, about 0.2% w/w of sodium citrate dihydrate, about 0.025% w/w of disodium Edetate, solvents and pH adjusting agents.

In embodiments of the invention, the present invention provides a topical pharmaceutical solution comprising of about 0.01% w/w to about 0.1% w/w of methotrexate, about 0.3% w/w of xanthan gum, about 0.3% w/w of tromethamine, about 0.2% w/w of sodium citrate dihydrate, about 0.025% w/w of disodium Edetate, solvents and pH adjusting agents to adjust the pH of about 7.0.

In still another embodiment of the invention, the present invention provides a topical pharmaceutical solution consisting essentially of about 0.01% w/w to about 0.1% w/w of methotrexate, about 0.3% w/w of xanthan gum, about 0.3% w/w of tromethamine, about 0.2% w/w of sodium citrate dihydrate, about 0.025% w/w of disodium Edetate, solvents and pH adjusting agents to adjust the pH of about 7.0.

In embodiments of the invention, there is provided a process for preparing the topical solution comprising the steps of

a) dissolving the polymer in the heated solvent to obtain the solution of the polymer;
b) dissolving the solubilizer in solvent to obtain the solution of solubilizer;
c) dissolving the methotrexate in solution of solubilizer to obtain a methotrexate solution;
d) Adding the buffering agent and antioxidant to the methotrexate solution and adjust the pH to about 7.0 with the pH adjusting agent to form the pH adjusted methotrexate solution;
e) Adding the pH adjusted methotrexate solution to the solution of polymer with stirring; and adjusting the pH to about 7.0 with the pH adjusting agent to obtain the topical solution.

In another embodiment of the invention, there is provided a process for preparing the topical solution comprising the steps of

a) dissolving of about 0.1% w/w to about 1.0% w/w of xanthan gum in the heated solvent to obtain the solution of the polymer;
b) dissolving the solubilizer in solvent to obtain the solution of solubilizer;
c) dissolving of about 0.01% w/w to about 0.1% w/w methotrexate in solution of solubilizer to obtain a methotrexate solution;
d) Adding the buffering agent and antioxidant to the methotrexate solution and adjust the pH to about 7.0 with the pH adjusting agent to form the pH adjusted methotrexate solution;
e) Adding the pH adjusted methotrexate solution to the solution of polymer with stirring; and adjusting the pH to about 7.0 with the pH adjusting agent to obtain the topical solution.

In still another embodiment of the invention, there is provided a process for preparing the topical solution comprising the steps of

a) dissolving of about 0.3% w/w of xanthan gum in the heated solvent to obtain the solution of the polymer;
b) dissolving the solubilizer in solvent to obtain the solution of solubilizer;
c) dissolving of about 0.01% w/w to about 0.1% w/w methotrexate in solution of solubilizer to obtain a methotrexate solution;
d) Adding the buffering agent and antioxidant to the methotrexate solution and adjust the pH to about 7.0 with the pH adjusting agent to form the pH adjusted methotrexate solution;
e) Adding the pH adjusted methotrexate solution to the solution of polymer with stirring; and adjusting the pH to about 7.0 with the pH adjusting agent to obtain the topical solution.

In embodiments of the invention, there is provided a process for preparing the topical solution comprising the steps of

a) dissolving the xanthan gum in the heated solvent to obtain the solution of the polymer;
b) dissolving the tromethamine in solvent to obtain the solution of solubilizer;
c) dissolving the methotrexate in solution of solubilizer to obtain a methotrexate solution;
d) Adding the sodium citrate dihydrate and disodium Edetate to the methotrexate solution and adjust the pH to about 7.0 with the pH adjusting agent to form the pH adjusted methotrexate solution;
e) Adding the pH adjusted methotrexate solution to the solution of xanthan gum with stirring; and adjusting the pH to about 7.0 with the pH adjusting agent to obtain the topical solution.

In another embodiment of the invention, there is provided a process for preparing the topical solution comprising the steps of

a) dissolving of about 0.1% w/w to about 1.0% w/w of xanthan gum in the heated solvent to obtain the solution of the polymer;
b) dissolving the tromethamine in solvent to obtain the solution of solubilizer;
c) dissolving of about 0.01% w/w to about 1.0% w/w of methotrexate in solution of solubilizer to obtain a methotrexate solution;
d) Adding the sodium citrate dihydrate and disodium Edetate to the methotrexate solution and adjust the pH to about 7.0 with the pH adjusting agent to form the pH adjusted methotrexate solution;
e) Adding the pH adjusted methotrexate solution to the solution of xanthan gum with stirring; and adjusting the pH to about 7.0 with the pH adjusting agent to obtain the topical solution.

In still another embodiment of the invention, there is provided a process for preparing the topical solution comprising the steps of

a) dissolving of about 0.3% w/w of xanthan gum in the heated solvent to obtain the solution of the polymer;
b) dissolving the tromethamine in solvent to obtain the solution of solubilizer;
c) dissolving of about 0.01% w/w to about 1.0% w/w of methotrexate in solution of solubilizer to obtain a methotrexate solution;
d) Adding the sodium citrate dihydrate and disodium Edetate to the methotrexate solution and adjust the pH to about 7.0 with the pH adjusting agent to form the pH adjusted methotrexate solution;
e) Adding the pH adjusted methotrexate solution to the solution of xanthan gum with stirring; and adjusting the pH to about 7.0 with the pH adjusting agent to obtain the topical solution.

In a further embodiment of the invention, there is provided a process for preparing the topical solution comprising the steps of

a) dissolving of about 0.3% w/w of xanthan gum in the heated solvent to obtain the solution of the polymer;
b) dissolving of about 0.3% w/w of tromethamine in solvent to obtain the solution of solubilizer;
c) dissolving of about 0.01% w/w to about 1.0% w/w of methotrexate in solution of solubilizer to obtain a methotrexate solution;
d) Adding of about 0.2% w/w of sodium citrate dihydrate and 0.025% w/w of disodium Edetate to the methotrexate solution and adjust the pH to about 7.0 with the pH adjusting agent to form the pH adjusted methotrexate solution;
e) Adding the pH adjusted methotrexate solution to the solution of xanthan gum with stirring; and adjusting the pH to about 7.0 with the pH adjusting agent to obtain the topical solution.

In embodiments of the invention, the present inventive composition is used for the treatment of psoriasis.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Examples 1

Topical solution with the following composition is prepared.

| S.No | Ingredients | % w/w |
|---|---|---|
| 1 | Methotrexate | 0.01%-0.5% |
| 2 | Tromethamine | 0.01%-5% |
| 3 | Xanthan gum | 0.01%-5% |
| 4 | Sod citrate dihydrate | 0.1%-0.3% |
| 5 | Disodium EDTA | 0.001%-0.3% |
| 6 | Sodium Hydroxide | Q.S to pH 7 |
| | (pH adjusting agent) | |
| 7 | Hydrochloric acid | Q.S to pH 7 |
| | (pH adjusting agent) | |
| 8 | Purified Water | Q.S to 100% |

These topical solution is prepared as follows:
1. Preparation of Polymer Phase:
   a. Take about 60% of purified water in suitable container and heat the purified water to 70° C.,
   b. Slowly add and dissolve the xanthan gum in above heated purified water and stir for about 60 minutes,
   c. After completely dissolving the polymer, allow to cool to room temperature.
2. Preparation of API Phase:
   a. Take about 20% of purified water and add Tromethamine,
   b. To above solution add and dissolve the methotrexate API,
   c. To above methotrexate solution add and dissolve Trisodium citrate dihydrate and disodium EDTA,
   d. Check the pH of the solution and adjust to 7.0 using sodium hydroxide solution or hydrochloric acid.
3. Addition of API Phase to Polymer Phase
   a. Slowly transfer the API phase to polymer phase and stir the solution,
   b. Check the pH, and adjust the pH of solution to 7.0 using sodium hydroxide solution or hydrochloric acid.
   4. Make up the volume to 100% using purified water and stir for 15 minutes to make the topical solution comprising methotrexate.

### Example 2

Topical solution with the following composition is prepared.

| S.No | Ingredients | % w/w |
|---|---|---|
| 1 | Methotrexate | 0.01%-0.5% |
| 2 | Tromethamine | 0.01%-5% |
| 3 | Xanthan gum | 0.01%-5% |
| 4 | Sod citrate dihydrate | 0.1%-0.3% |
| 5 | Disodium EDTA | 0.001%-0.3% |
| 6 | Methyl Paraben | Q.S |
| 7 | Propyl Paraben | Q.S |
| 8 | Sodium Hydroxide | Q.S to pH 7 |
| | (pH adjusting agent) | |
| 9 | Hydrochloric acid | Q.S to pH 7 |
| | (pH adjusting agent) | |
| 10 | Purified Water | Q.S to 100% |

The topical solution is prepared as follows:
1. Preparation of Polymer Phase:
   a. Take about 60% of purified water in suitable container and heat the purified water to 70° C.,
   b. Add and dissolve methyl paraben and propyl paraben to the above heated purified water to get a clear solution,
   c. Slowly add and dissolve the xanthan gum in the contents of step b and stir for about 60 minutes,
   d. After completely dissolving the polymer, allow to cool to room temperature.
2. Preparation of API Phase:
   a. Take about 20% of purified water and add Tromethamine,
   b. To above solution add and dissolve the methotrexate API,
   c. To above methotrexate solution add and dissolve Trisodium citrate dihydrate and disodium EDTA,
   d. Check the pH of the solution and adjust to 7.0 using sodium hydroxide solution or hydrochloric acid.
3. Addition of API Phase to Polymer Phase
   a. Slowly transfer the API phase to polymer phase and stir the solution,
   b. Check the pH, and adjust the pH of solution to 7.0 using sodium hydroxide solution or hydrochloric acid.
4. Make up the volume to 100% using purified water and stir for 15 minutes to make the topical solution comprising methotrexate.

### Example 3 to 6

| | | | | | Ex. 6 |
|---|---|---|---|---|---|
| | | Ex. 3 | Ex. 4 | Ex. 5 | % w/w |
| S.No | Ingredients | % w/w | % w/w | % w/w | (Placebo) |
| 1 | Methotrexate | 0.1% | 0.5% | 1.0% | — |
| 2 | Tromethamine | 0.3% | 0.3% | 0.3% | 0.3% |
| 3 | Xanthan gum | 1.0% | 1.0% | 1.0% | 1.0% |
| 4 | Sod citrate dihydrate | 0.2% | 0.2% | 0.2% | 0.2% |
| 5 | Disodium EDTA | 0.025% | 0.025% | 0.025% | 0.025% |
| 6 | Sodium Hydroxide | Q.S to | Q.S to | Q.S to | Q.S to |
| | (pH adjusting agent) | pH 7 | pH 7 | pH 7 | pH 7 |
| 7 | Hydrochloric acid | Q.S to | Q.S to | Q.S to | Q.S to |
| | (pH adjusting agent) | pH 7 | pH 7 | pH 7 | pH 7 |
| 8 | Purified Water | Q.S to | Q.S to | Q.S to | Q.S to |
| | | 100% | 100% | 100% | 100% |

The topical solution is prepared by the process as disclosed in Example 1.

### Example 7: Effect of Methotrexate Formulation in Imiquimod Induced Psoriasis in Balb/c Mice Model

Hair of Balb/c mice was removed from the dorsal area of skin by trimming and marking the region. After hair removal animals were randomized and divided into seven groups and 62.5 mg of Imiquimod (IMQ) cream was weighed and gently applied to each shaved and marked region of animals for induction of psoriasis.
1^{st} group (control group)—no application of any formulation
2^{nd} group (positive control (indication group)—Imiquimod cream)—application of Imiquimod cream
3^{rd} group (only placebo formulation as in Example 6)—application of placebo formulation as in Example 6 once weekly 10.25 mg.
4^{th} group (Example 3 formulation with 0.1% methotrexate)—example 3 formulation applied to dorsal area of ear skin once weekly 10.25 mg.
5^{th} group (Example 4 formulation with 0.5% methotrexate)—example 4 formulation applied to dorsal area of ear skin once weekly 10.25 mg.
6^{th} group (Example 5 formulation with 1.0% methotrexate)—example 5 formulation applied to dorsal area of ear skin once weekly 10.25 mg.
7^{th} group (Reference Folitrax Methotrexate Injection 25 mg/mL)—5.125 μg/kg/week administration.

After treatment, the skin reactions were scored on each day and scoring of erythema (psoriatic scoring) was done based the grading scale as mentioned below.

| Skin reaction | Grading scale |
|---|---|
| No lesion | 0 |
| Slightly Pink | 1 |
| Pink | 2 |
| Red | 3 |
| Dark Red | 4 |

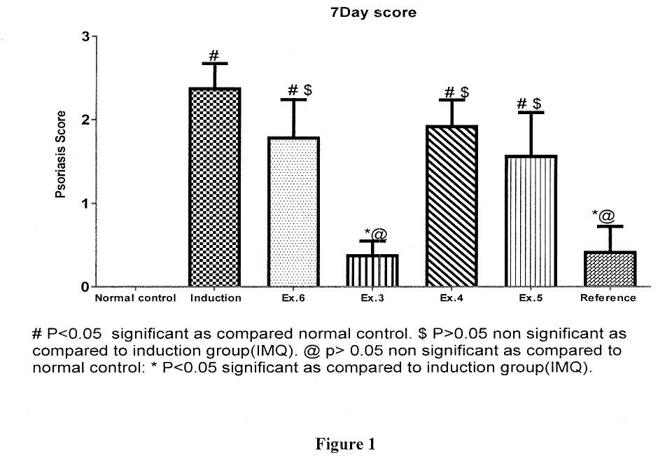

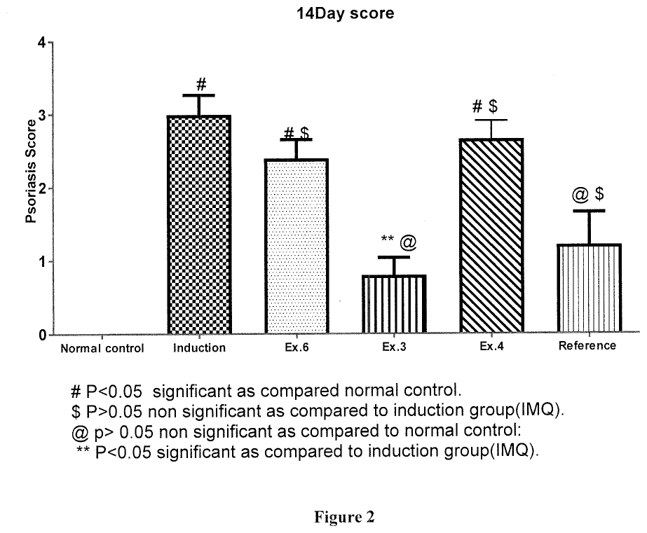

The score on 7 days and 14 days after the treatment was disclosed in FIG. 1 and FIG. 2 respectively. The results yielded that only methotrexate topical formulation with 0.1% of methotrexate (example 3 formulation) has shown significant results (P<0.050) when compared to the positive control (induction group).

### Example 8 to 11

| | | Ex. 8 | Ex. 9 | Ex. 10 | Ex. 11 |
|---|---|---|---|---|---|
| S.No | Ingredients | % w/w | % w/w | % w/w | % w/w |
| 1 | Methotrexate | 0.1% | 0.1% | 0.1% | 0.1% |
| 2 | Tromethamine | 0.3% | 0.3% | 0.3% | 0.3% |
| 3 | Xanthan gum | 0.1% | 0.3% | 0.5% | 1.0% |
| 4 | Sod citrate dihydrate | 0.2% | 0.2% | 0.2% | 0.2% |
| 5 | Disodium EDTA | 0.025% | 0.025% | 0.025% | 0.025% |
| 6 | Sodium Hydroxide | Q.S to | Q.S to | Q.S to | Q.S to |
| | (pH adjusting agent) | pH 7 | pH 7 | pH 7 | pH 7 |
| 7 | Hydrochloric acid | Q.S to | Q.S to | Q.S to | Q.S to |
| | (pH adjusting agent) | pH 7 | pH 7 | pH 7 | pH 7 |
| 8 | Purified Water | Q.S to | Q.S to | Q.S to | Q.S to |
| | | 100% | 100% | 100% | 100% |

The topical solution is prepared by the process as disclosed in Example 1.

### Example 12: Effect of Xanthan Gum Polymer Concentration on In Vitro Permeation on Pork Skin

In vitro permeation study on pork skin of topical solutions of examples 8, 9, 10 and 11 containing 0.1%, 0.3%, 0.5% and 1.0% w/w of xanthan gum respectively with 0.1% of methotrexate was conducted to determine flux and the results are disclosed in Table 1.

TABLE 1

| | Time/Flux (μg/cm²/hr) | | | | | | |
|---|---|---|---|---|---|---|---|
| | | | | | | | Average |
| | 1 hr | 2 hr | 4 hr | 6 hr | 18 hr | 24 hr | Flux |
| Ex. 8 Topical solution | 0.50 | 1.10 | 1.75 | 2.05 | 2.95 | 4.45 | 2.13 |
| (0.1% Xanthan gum) | | | | | | | |
| Ex. 9 Topical solution | 0.20 | 0.80 | 1.00 | 1.60 | 1.63 | 2.13 | 1.23 |
| (0.3% Xanthan gum) | | | | | | | |
| Ex. 10 Topical solution | 0.10 | 0.40 | 0.45 | 0.70 | 0.87 | 1.63 | 0.69 |
| (0.5% Xanthan gum) | | | | | | | |
| Ex. 11 Topical solution | 0.10 | 0.40 | 0.60 | 0.15 | 1.05 | 1.27 | 0.59 |
| (1% Xanthan gum) | | | | | | | |

Based on the above results of flux determination in Table 1, methotrexate permeation decreased with the increase of polymer (xanthan gum) concentration. The 0.3% of xanthan gum was selected as polymer concentration as it has the optimal flux and ease of application (patient acceptability) properties, reduction in toxicity by required release of drug with optimal flux.

### Example 13 to 16

| | | | | | Ex. 16 |
|---|---|---|---|---|---|
| | | Ex. 13 | Ex. 14 | Ex. 15 | % w/w |
| S.No | Ingredients | % w/w | % w/w | % w/w | (placebo) |
| 1 | Methotrexate | 0.01% | 0.05% | 0.1% | — |
| 2 | Tromethamine | 0.3% | 0.3% | 0.3% | 0.3% |
| 3 | Xanthan gum | 0.3% | 0.3% | 0.3% | 0.3% |
| 4 | Sod citrate dihydrate | 0.2% | 0.2% | 0.2% | 0.2% |
| 5 | Disodium EDTA | 0.025% | 0.025% | 0.025% | 0.025% |
| 6 | Sodium Hydroxide | Q.S to | Q.S to | Q.S to | Q.S to |
| | (pH adjusting agent) | pH 7 | pH 7 | pH 7 | pH 7 |
| 7 | Hydrochloric acid | Q.S to | Q.S to | Q.S to | Q.S to |
| | (pH adjusting agent) | pH 7 | pH 7 | pH 7 | pH 7 |
| 8 | Purified Water | Q.S to | Q.S to | Q.S to | Q.S to |
| | | 100% | 100% | 100% | 100% |

The topical solution is prepared by the process as disclosed in Example 1.

### Example 17: Effect of Methotrexate Formulation in Imiquimod Induced Psoriasis in Balb/c Mice Model

Hair of Balb/c mice was removed from the dorsal area of skin by trimming and marking the region. After hair removal animals were randomized and divided into seven groups and 62.5 mg of Imiquimod (IMQ) cream was weighed and gently applied to each shaved and marked region of animals for induction of psoriasis.
1^{st} group (control group)—no application of any formulation
2^{nd} group (positive control (indication group)—Imiquimod cream)—application of Imiquimod cream
3^{rd} group (Reference group—Clobetasol propionate topical solution 0.05% w/w)—twice daily at 14.57 μl.
4^{th} group (Example 13 formulation with 0.01% methotrexate)—example 13 formulation applied to dorsal area of ear skin once weekly 10.25 mg.
5^{th} group (Example 14 formulation with 0.05% methotrexate)—example 14 formulation applied to dorsal area of ear skin once weekly 10.25 mg.
6^{th} group (Example 15 formulation with 0.1% methotrexate)—example 15 formulation applied to dorsal area of ear skin once weekly 10.25 mg.
7^{th} group (placebo formulation as in Example 16)—application of placebo formulation once weekly 10.25 mg.

After treatment, the skin reactions were scored on each day and scoring of erythema (psoriatic scoring) was done based the grading scale as mentioned below.

| Skin reaction | Grading scale |
|---|---|
| No lesion | 0 |
| Slightly Pink | 1 |
| Pink | 2 |
| Red | 3 |
| Dark Red | 4 |

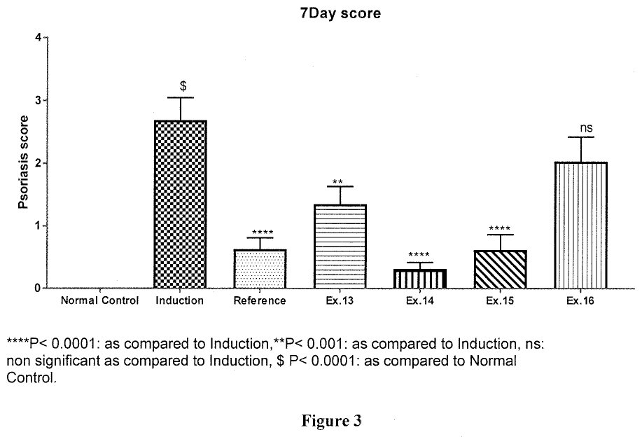

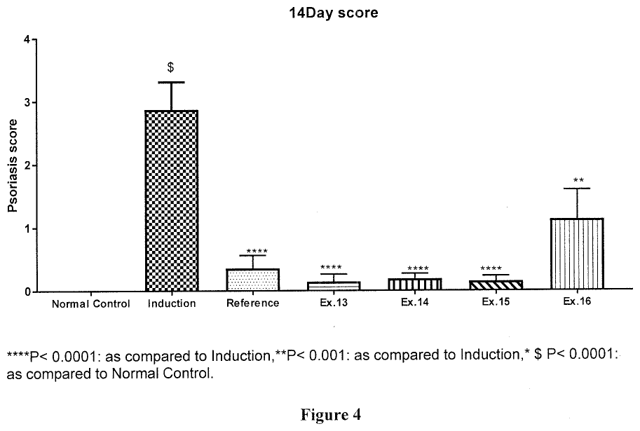

The score on 7 days and 14 days after the treatment was disclosed in FIG. 3 and FIG. 4 respectively. The results yielded that methotrexate formulation with 0.01% w/w, 0.05% w/w and 0.1% w/w of methotrexate of example 13, 14 and 15 respectively have shown significant treatment results (P<0.0001) when compared to the positive control (induction group).

1. A topical pharmaceutical composition comprising of about 0.01% w/w to about 0.1% w/w of methotrexate, about 0.1% w/w to about 1.0% w/w of xanthan gum and pharmaceutically acceptable excipients.

2. The composition of claim 1, wherein the composition comprises of about 0.3% w/w of xanthan gum.

3. The composition of claim 1, wherein pharmaceutically acceptable excipients are selected from group comprising of solubilizers, buffering agents, antioxidants, preservatives, solvents and pH adjusting agents to adjust the pH of about 7.0.

4. The composition of claim 3, wherein solubilizers are selected from the group consisting of tromethamine, glyceryl oleate, isopropyl myristate, methyl laurate, N-lauroyl sarcosine, oleic acid (octadecenoic acid), sodium lauryl sulfoacetate, sodium octyl sulfate, tromethamine and propylene glycol.

5. The composition of claim 3, wherein buffering agents are selected from group consisting of diethanolamine, triethanolamine, sodium hydroxide, hydrochloric acid, sodium citrate dihydrate, citric acid and mono basic sodium phosphate.

6. The composition of claim 3, wherein antioxidants are selected from group consisting of butylated hydroxyanisole (BHA), butylated hydroxytoluene (BHT), ascorbic acid and disodium Edetate (disodium EDTA).

7. The composition of claim 4, wherein the composition comprises of about 0.3% w/w of tromethamine.

8. The composition of claim 5, wherein the composition comprises of about 0.2% w/w of sodium citrate dihydrate.

9. The composition of claim 6, wherein the composition comprises of about 0.025% w/w of disodium EDTA.

10. A topical pharmaceutical solution comprising of about 0.01% w/w to about 0.1% w/w of methotrexate, about 0.3% w/w of xanthan gum, about 0.3% w/w of solubilizers, about 0.2% w/w of buffering agents, about 0.025% w/w of antioxidants, solvents and pH adjusting agents to adjust the pH of about 7.0.
